(19) United States
(12) Patent Application Publication
(10) Pub. No.: US 2025/0027904 A1
(43) Pub. Date: Jan. 23, 2025

(54) METHOD FOR DETERMINING THE CONTENT OF AT LEAST METALLIC IRON IN SPONGE IRON - OR A SAMPLE THEREOF - THAT IS PRODUCED BY DIRECT REDUCTION FROM IRON ORE

(71) Applicant: VOESTALPINE STAHL GMBH, Linz (AT)

(72) Inventors: Stefan SCHUSTER, Enns (AT); Stefan SCHEIBLHOFER, Enns (AT); Norbert GSTÖTTENBAUER, Engerwitzdorf (AT); Daniel WÖCKINGER, Linz (AT); Christopher HARRIS, Linz (AT); Wolfgang AMRHEIN, Linz (AT); Christoph THALER, Linz (AT); Anton PICHLER, Zeillern (AT); Anna Sonja GRIESSER, Kirchdorf an der Krems (AT); Joachim MAUHART, Waldneukirchen (AT); Christoph FEILMAYR, Linz (AT); Erfried GANGL, Hofkirchen (AT)

(21) Appl. No.: 18/710,378

(22) PCT Filed: Nov. 15, 2022
(86) PCT No.: PCT/EP2022/082048

§ 371 Date: May 15, 2024

(30) Foreign Application Priority Data
Nov. 15, 2021 (EP) 21208339.8

(51) Int. Cl. G01N 27/72; G01N 33/2028

(52) U.S. Cl. CPC G01N 27/72; G01N 33/2028

(57) ABSTRACT

A method for determining the content of at least metallic iron (Feₘₑₜ) in sponge iron or a sample thereof produced by direct reduction from iron ore. For this purpose, a mathematical model is provided, whose model output variable (A_{mod}) is described as a function of the content of at least metallic iron (Feₘₑₜ) of the sponge iron or sample thereof, this mathematical model having an effective medium approximation (EMA) for the permeability (μ_{eff}) and electrical conductivity (σ_{eff}) of the sponge iron or sample thereof.

### TECHNICAL FIELD

The invention relates to a method for determining the content of at least metallic iron in sponge iron—or a sample thereof—that is produced by direct reduction from iron ore.

### PRIOR ART

To determine the degree of metallization of sponge iron or a sample thereof, namely a measuring volume of the sponge iron, it is known from the prior art (DE3017001A1) to detect a measurement variable, namely the impedance of a measuring coil, which is coupled to an excitation coil via the sponge iron or a sample thereof. The excitation coil impresses magnetic fields, which change over time and have different frequencies from one another, into the sponge iron or a sample thereof. The measurement variable therefore depends on at least one electromagnetic property of the sponge iron or portion thereof. According to DE3017001A1, the impedance of the measuring coil should have a close relationship to the bulk conductivity of the sponge iron or sample thereof and thus to its degree of metallization. A disadvantage of this method is the comparatively high inaccuracy in the relationship between the measurement data and bulk conductivity, which allows only a rough estimate of the degree of metallization. Consequently, such a method is also unsuitable for an exact control of a direct reduction process, for example.

### SUMMARY OF THE INVENTION

The object of the invention, therefore, is to improve the accuracy of a method for determining the content of at least metallic iron in sponge iron.

The accuracy of the method can be significantly increased if not only—as disclosed in the prior art—is a relationship between a detected electrical measurement variable and an electromagnetic property of the sponge iron used to determine the content of at least one element of the sponge iron or sample thereof, but also a mathematical model is used for this purpose. More particularly according to the invention, this is done by providing a mathematical model whose model output variable is described as a function of the content of at least metallic iron (Feₘₑₜ) of the sponge iron or a sample thereof, this mathematical model having an effective medium approximation (EMA) for the permeability (μ_{eff}) and electrical conductivity (σ_{eff}) of the sponge iron or sample thereof. Specifically, it has surprisingly turned out that with an effective medium approximation (EMA) for the permeability and electrical conductivity, the content determination can be carried out much more accurately and/or more quickly if an estimation method for determining the content of at least metallic iron (Feₘₑₜ) is carried out using the detected measurement variable and the mathematical model. The fast and accurate method according to the invention is therefore also particularly suitable for monitoring, regulating, or controlling a process for direct reduction of iron ore.

Preferably, the mathematical model includes a gangue fraction (p_{g}) of the iron ore or sponge iron or sample thereof as a first input parameter and/or a density (ρₜₒₜ), more particularly an effective density, of the sponge iron or sample thereof as a second input parameter.

If the gangue fraction is specified as the first input parameter, then inaccuracies in the content determination can be further reduced since this step allows the iron-containing fractions (metallic iron, iron oxides, iron carbide) in the solids fraction of the measured volume to be determined much more accurately. Preferably, the gangue fraction can be a variable input parameter that results from the iron ore used and that can be used in the mathematical model. This can further increase the accuracy of the method.

The density of the sponge iron or a sample thereof as a second input parameter can be determined easily from a process standpoint, for example, by weighing or on the basis of the iron ore used. It is thus possible, for example, to precisely determine the gaseous fraction and—since both the magnetic and electrical parameters of the gas fraction are known—the content determination can be carried out particularly accurately and robustly with respect to interfering influences. Preferably, this density can be a variable input parameter that results from the iron ore used and that can be used in the mathematical model. This can further increase the accuracy of the method.

If the mathematical model is an electromagnetic model (EMM), then the accuracy of the method can be further increased. This is the case more particularly because the modeled variable can more accurately represent the electrical measurement variable.

If the model output variable A_{mod} is an electrical impedance Z_{mod}(ωᵢ), then the fractions of magnetic and non-magnetic substances/materials such as metallic iron Feₘₑₜ, iron oxide FeO, and iron carbide Fe₃C can be determined more accurately.

Preferably, the impedance model has a modeling of at least one electrical coil with sponge iron or a sample thereof as the material of the core.

If an electrical voltage U_{mod}(ωᵢ) is modeled, which forms taking into account a known current, more particularly an impressed current, at the electrical impedance of the impedance model or at the modeled impedance, then the model parameters of the fractions of magnetic and non-magnetic materials such as iron and/or iron oxide and/or iron carbide can be determined, which can further increase the accuracy of the method.

Alternatively, it is conceivable to model an electrical current I_{mod}(ωᵢ) that occurs when a known voltage, more particularly an impressed voltage, is applied at the electrical impedance of the impedance model or at the modeled impedance.

By contrast with the prior art, a particularly accurate content determination is made possible if the estimation method is used to determine the content or contents for which the deviation of the at least one detected measurement variable (Aₘₑₐₛ) from the model output variable (A_{mod}) of the mathematical model is minimal. Preferably, the estimation method is used to determine the content or contents for which the deviation of the sum of the detected measurement variables (Aₘₑₐₛ) from the model output variable (A_{mod}) of the mathematical model is minimal.

This estimation method specifically makes it possible to create an extremely sensitive stochastic method—which simplifies the task of collecting measurement data. For example, simply applying two magnetic fields can enable acquisition of enough measurement data to permit extremely accurate conclusions to be drawn about the content of metallic iron and/or iron oxide and/or iron carbide in the sponge iron. More particularly, a least squares estimation method can be suitable for this purpose.

Preferably, the induced electrical voltage Uₘₑₐₛ in a coil, which results from the magnetic field generated by an impressed current I, is detected as or for the measurement variable Aₘₑₐₛ, wherein the coil has sponge iron or a sample thereof as the material of the core.

Alternatively, it is conceivable for the electrical current Iₘₑₐₛ, which occurs in the coil when an impressed voltage U is applied to this coil, to be detected as or for the measurement variable Aₘₑₐₛ, wherein the coil has sponge iron or a sample thereof as the material of the core.

The accuracy of the method can be further improved if first measurement data of the measurement variable are detected by inducing a first magnetic field into the sponge iron or a sample thereof and second measurement data of the measurement variable are detected by inducing a second magnetic field into the sponge iron or a sample thereof, wherein the magnetic fields change over time and have different frequencies from each other. The accuracy can be further increased by detecting additional measurement data at additional frequencies.

Preferably, the model output variable (A_{mod}) can be described as a function of the content of at least metallic iron (Feₘₑₜ) and at least one iron oxide (Fe₂O₃, Fe₃O₄, or FeO) as well as iron carbide (Fe₃C) of the sponge iron or a sample (4) thereof. As a result, the estimation method for determining the content of at least metallic iron (Feₘₑₜ), iron oxide (Fe₂O₃, Fe₃O₄, or FeO), and iron carbide (Fe₃C) can be carried out using the detected measurement variable (Aₘₑₐₛ) and the mathematical model.

### BRIEF DESCRIPTION OF DRAWINGS

The subject of the invention is shown in greater detail in the drawings based on an exemplary embodiment. In the drawings:

FIG. 1 shows a simplified depiction of a mathematical model 1 and

FIG. 2 shows a schematic representation of a measurement setup.

### DETAILED DESCRIPTION OF THE INVENTION

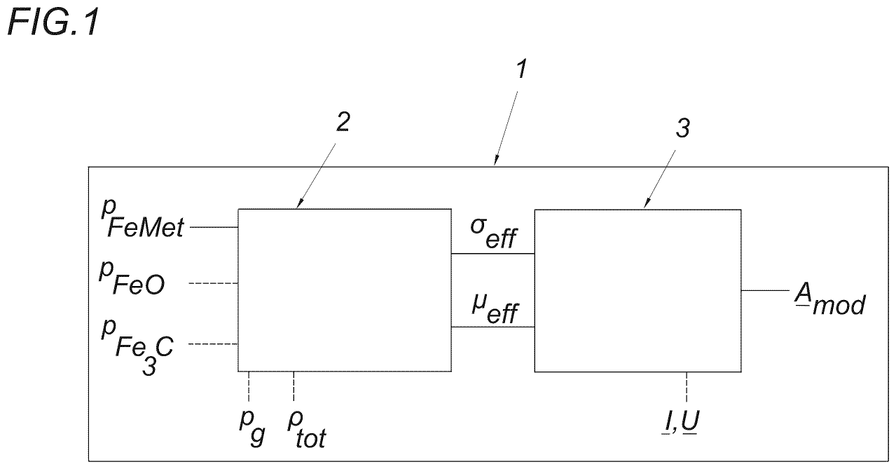

The mathematical model 1 shown in FIG. 1 is used together with an electrical measurement variable Uₘₑₐₛ for determining the content p_{FeMet} of metallic iron Feₘₑₜ, for determining the content p_{FeO} of iron oxide FeO, and for determining the content p_{Fe3C} of iron carbide Fe₃C.

The mathematical model 1 describes the electromagnetic properties of the sponge iron or a sample 4 thereof as a function of the content p_{FeMet} of metallic iron Feₘₑₜ, the content p_{FeO} of iron oxide FeO, and the content p_{Fe3C} of iron carbide Fe₃C. In each case pᵢ where i={FeMet, Fe0, Fe₃0₄, . . . } is the mass in relation to the total mass of the material in the sample container, for example p_{FeMet} is the mass of metallic iron Feₘₑₜ in relation to the total mass of the material in the sample container.

In addition to this, the masses of iron oxides and iron carbides are often also of interest when estimating the quality of directly reduced iron. Due to the composition of the raw material used, gangue is also present in the directly reduced material. In summary, the material to be analyzed consists of metallic iron Feₘₑₜ, iron oxides, and iron carbides with a mass fraction pᵢ of the total mass and of fractions of non-ferrous materials combined into gangue with a mass fraction p_{g} of the total mass.

This gangue fraction p_{g} can be determined using a wide variety of methods (e.g. chemical analyses) or if the gangue fraction p_{g} is known to be a typical characteristic for raw material from certain mining areas, then the mass of the gangue can be determined, making it possible to calculate the total mass of iron, iron carbides, and iron oxides within the volume. Finally, a mathematical model must be used to separate out the fraction of metallic iron from the remaining mass. According to the invention, this is done using the electromagnetic properties of the different ingredients.

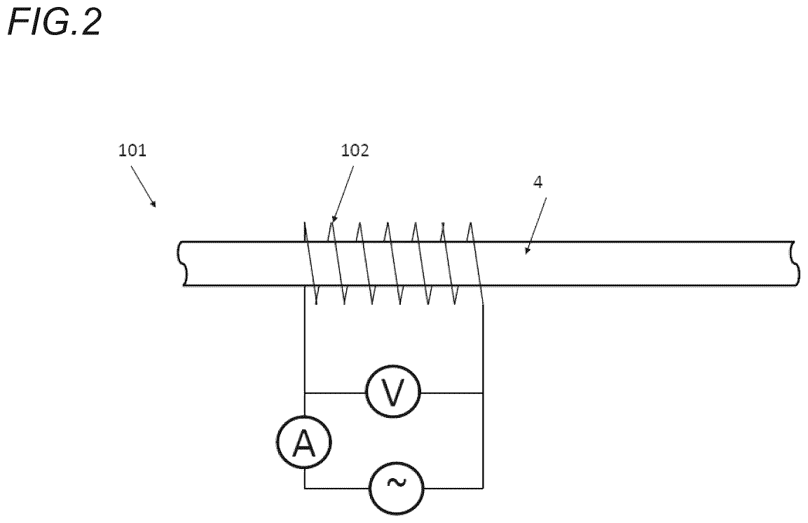

In a preferred variant, the measurement setup 100 shown in FIG. 2 comprises a coil 101 in the form of a cylindrical coil, which encloses the sponge iron or a sample 4 thereof and thus uses it as a core material. As an alternative to the coil shown in FIG. 2, one or more excitation coils can also be used in combination with one or more measuring coils, which is not shown in detail.

This measurement setup results in a simplified impedance model 3, which is derived from an analytical description of the impedance of a homogeneous cylinder whose electromagnetic properties are influenced by the iron content.

The relationship between the measurement variable Uₘₑₐₛ and the magnetic properties of the sample 4 results from the solution of the magnetic circuit 4, i.e. in addition to the electrical and magnetic properties of the sample 4, the geometry of the setup and the electrical properties of the measurement setup (such as the diameter of the cylindrical coil 101 and the resistance of the coil 101) are also taken into account. This basic procedure will be explained in greater detail below.

According to one exemplary embodiment, the sample material is placed in a narrow, cylindrical, non-magnetic, electrically non-conductive container. A short ring-shaped coil 101 is positioned around this container, which can function both as an excitation coil and as a measuring coil.

For the modeling of an impedance model 3, it can be assumed for the modeled impedance Z of this exemplary embodiment of a measuring apparatus that the sample volume is an approximately infinitely long cylinder in relation to the height of the measuring coil and that the measuring system is a cylindrical coil encompassing the sample volume (see H. Libby, Introduction to Electromagnetic Nondestructive Test Methods, Wiley, 1971). This means that the impedance Z of the measurement system can be modeled using a mathematical electromagnetic model (EMM)

$$\underline{Z} = ω {μ}_{eff} π {a}^{2} [ \frac{2}{ka} \frac{{ber}^{′} ( k * a ) + j bei ′ ( k * a )}{ber ( k * a ) + j bei ( k * a )} ] + j ω {μ}_{eff} π ( {b}^{2} - {a}^{2} )$$

where
ω is the circular frequency of the impressed alternating current
μ_{eff} is the effective permeability of the sample volume
a is the average radius of the sample volume
b is the average radius of the cylindrical coil
ber(k*a) is the real part of the Kelvin-Bessel function
ber′(k*a) is the 1^{st} derivative of ber(k*a)
bei(k*a) is the imaginary part of the Kelvin-Bessel function
bei′(k*a) is the 1^{st} derivative of bei(k*a)
k is the characteristic number of the eddy current problem
and

$$k = \sqrt{ω {μ}_{eff} {σ}_{eff} + j {ω}^{2} {μ}_{eff} {ε}_{0}}$$

where
ω is the circular frequency of the impressed alternating current
μ_{eff} is the effective permeability of the sample volume
σ_{eff} is the electrical conductivity of the sample volume
∈₀ is the electric field constant (8.854 10⁻¹² As/Vm).

In order to complete the mathematical model 1 for the content determination and thus to obtain the material breakdown, the mathematical model 1 has an effective medium approximation (EMA) 2 for the effective permeability μ_{eff} and effective electrical conductivity σ_{eff} of the sponge iron or a sample 4 thereof. The EMA 2 basically establishes a mathematical relationship between effective material properties and the individual constituents and their volume fractions.

For the sake of simplicity in this exemplary embodiment, the sample 4 is assumed to consist of two material phases: one the one hand, metallic iron Feₘₑₜ, a ferromagnetic material with good conductivity, and on the other hand, the remaining components, weakly ferromagnetic material with very poor or non-existent conductivity. It is therefore assumed here that the metallic iron Feₘₑₜ with a volume fraction f_{FeMet} represents the ferromagnetic material with good conductivity, and iron oxides, gangue, and air all together represent the weakly ferromagnetic material with comparatively poor conductivity.

For example, an “inverse Maxwell-Garnett formula” for two material phases is sufficient for determining the fraction f_{FeMet} of the metallic iron Feₘₑₜ in relation to the volume of the sample volume according to this exemplary embodiment (see J. C. Maxwell-Garnett, Colours in Metal Glasses and in Metal Films, Trans. Royal Soc. London, vol. 203, pp. 385-420, 1904)

$${μ}_{eff} = {μ}_{FeMet} + 3 ( 1 - {f}_{FeMet} ) {μ}_{FeMet} \frac{{μ}_{res} - {μ}_{FeMet}}{{μ}_{res} + 2 {μ}_{FeMet} - ( 1 - {f}_{FeMet} ) ( {μ}_{res} - {μ}_{FeMet} )}$$

where
μ_{FeMet} is the known permeability of pure iron (In the frequency range up to 100 Hz, this is approx. 5000 for magnetic flux densities below 0.9 T),
μᵣₑₛ is the unknown permeability of the non-ferromagnetic material
μ_{eff} is the resulting effective permeability of the material
f_{FeMet} is the sought volumetric content of metallic iron Feₘₑₜ

The effective electrical conductivity σ_{eff} of the material is modeled in a similar way. In this case, the inverse Maxwell-Garnett formula for two material phases is

$${σ}_{eff} = {σ}_{FeMet} + 3 ( 1 - {f}_{FeMet} ) {σ}_{FeMet} \frac{{σ}_{res} - {σ}_{FeMet}}{{σ}_{res} + 2 {σ}_{FeMet} - ( 1 - {f}_{FeMet} ) ( {σ}_{res} - {σ}_{FeMet} )}$$

where
σ_{FeMet} is the known electrical conductivity of pure iron (In the frequency range up to 100 Hz, this is approx. 10 Sm/mm².)
σᵣₑₛ is the electrical conductivity of the non-ferromagnetic material with a comparatively poor conductivity.

In a further simplification, this conductivity can be assumed to be zero.
σ_{eff} is the resulting effective electrical conductivity of the material
f_{FeMet} is the sought volumetric fraction of metallic iron

Depending on the type of raw material, a wide variety of EMAs—due to their material mixture, spatial distribution, and geometric proportions—are suitable for modeling two-phase and multi-phase material mixtures. For example, if not only the fraction p_{FeMet} of metallic iron Feₘₑₜ, but also the fraction of other metallic or electrically conductive materials, more particularly iron oxides and/or iron carbides, are to be determined, then a modeling approach for multiphase material mixtures can be selected for this purpose (e.g.: A. H. Sihvola and I. V. Lindell, Effective Permeability of Mixtures, Prog. Electromagn. Res. 06, pp. 153-180, 1992).

Typically, these formulas do not use the mass-related contents pᵢ of the individual ingredients to estimate the effective electromagnetic parameters, but rather the volume-related parameters fᵢ. The conversion can be made using the material density and the total effective density of the sample container σₜₒₜ.

$${f}_{i} = \frac{{ρ}_{tot}}{{ρ}_{i}} {p}_{i}$$

In mathematical model 1, the effective density ρₜₒₜ and the gangue fraction p_{g} of the sample 4 can be modeled as input parameters. This is done, for example, by using usually known values of the base material used. However, the gangue fraction p_{g} and the relative density ρₜₒₜ in the sample container can just as easily be determined precisely by calculating them using the previously determined mass of the material of the sample 4 and the known volume of the sample container.

Using the EMA equations for the effective permeability of the sample volume (μ_{eff}) and the electrical conductivity of the sample volume (σ_{eff}), with two unknowns, the permeability of the non-ferromagnetic material (μᵣₑₛ) and the sought volumetric fraction f_{FeMet} of metallic iron Feₘₑₜ, it is possible to determine the volumetric fraction of metallic iron Fe f_{FeMet} of metallic iron Feₘₑₜ of the sponge iron or sample 4.

Since the different models cannot be explicitly inserted into one another, especially in the case of multiphase material mixtures, a numerical estimation method can be used to solve the overall model. One possible method is, for example, a least squares estimation method. A least squares estimation method can be used to determine the metallic iron contents (Feₘₑₜ) for which the deviation of the modeled variable A_{mod} and the electrical measurement variable Aₘₑₐₛ is at a minimum. This also applies to the iron oxide (FeO) optionally shown in FIG. 1 as well as to the iron carbide (Fe₃C) optionally shown in FIG. 1 of the sample 4 in a corresponding mathematical model 1; the optional parts in FIG. 1 are identifiable by the dashed lines used to depict them.

In other words, if the complex impedance is known, then the effective permeability of the material and the effective electrical conductivity of the material can be calculated, which in turn can be used to calculate the volumetric composition by implementation of an EMA.

The complex impedance of the measuring system can in turn be determined from the measured current when a defined voltage is applied or from the measured voltage when a defined current is applied.

More particularly, the induced voltage in a coil (Uₘₑₐₛ, U_{mod}), the complex impedance of the measuring apparatus (Zₘₑₐₛ, Z_{mod}), or also the current through a measurement or excitation coil (Iₘₑₐₛ, I_{mod}) are suitable as measurement output variables (Aₘₑₐₛ) and model output variables (A_{mod}) for carrying out the measurement procedure.

In a preferred embodiment, an impedance is modeled as a model output variable A_{mod}, i.e. A_{mod} is an impedance Z_{mod}. In this case, the steps of the method are as follows in sequence:

a. Provision of a mathematical model. The model describes the impedance Z_{mod}(ω) of a sample 4 of a sponge iron in a measuring circuit as a function of the electrical frequency ω and as a function of the content of metallic iron (Feₘₑₜ), iron oxides (Fe₂O₃, Fe₃O₄, or FeO), and iron carbide (Fe₃C) of the sponge iron or sample 4 thereof.

For this description, the mathematical model 1 on the one hand has a determination of the impedance as a function of the permeability (μ_{eff}) and electrical conductivity (μ_{eff}) of the sponge iron or sample 4 thereof.

On the other hand, the model has a determination of the permeability (μ_{eff}) and electrical conductivity (σ_{eff}) as a function of the content of metallic iron (Feₘₑₜ), iron oxides (Fe₂O₃, Fe₃O₄, or FeO), and iron carbide (Fe₃C) of the sponge iron, and as a function of the gangue fraction and the density, for which an effective medium approximation (EMA) is implemented.

b. Insertion of the effective density ρₜₒₜ of the sample 4 and the gangue fraction p_{g} of the sample 4 into the mathematical model 1.

c. Introduction of a sample 4 of the sponge iron into the magnetic circuit of the measuring apparatus.

d. Impression of a first current curve I(ω₁) into the excitation coil together with determination/detection of the impedance Zₘₑₐₛ(ω₁) as a measurement variable (Aₘₑₐₛ) from the measurement of a first voltage curve Uₘₑₐₛ(ω₁) in the measuring coil.

e. Impression of a second current curve I(ω₂) into the excitation coil together with determination/detection of the impedance Zₘₑₐₛ(ω₂) as a measurement variable (Aₘₑₐₛ) from the measurement of a second voltage curve Uₘₑₐₛ(ω₂) in the measuring coil.

f. Optional: Impression of additional current curves I(ωᵢ) into the excitation coil together with determination/detection of the impedances Zₘₑₐₛ(ωᵢ) as measurement variables (Aₘₑₐₛ) from the measurements of additional voltage curves Uₘₑₐₛ(ωᵢ) in the measuring coil.

g. Determination of the fractions of metallic iron (Feₘₑₜ) and/or iron oxide (Fe₂O₃, Fe₃O₄, or FeO) and/or iron carbide (Fe₃C) in the sample 4 of sponge iron through implementation of a least squares estimation method using the mathematical model 1 and the measurement data for the voltage curves Uₘₑₐₛ(ω₁), Uₘₑₐₛ(ω₂), and optionally Uₘₑₐₛ(ωᵢ).

In this case, the fractions of metallic iron (Feₘₑₜ) and/or iron oxide (Fe₂O₃, Fe₃O₄, and FeO) and/or iron carbide (Fe₃C) in the sample 4 are determined for which the sum of the deviations of the modeled impedances Z_{mod} serving as the model output variable (A_{mod}) from the measured impedances Zₘₑₐₛ serving as the measurement variable (Aₘₑₐₛ) is minimal.

In an alternative embodiment, a voltage curve U(ωᵢ) can also be impressed into an excitation coil in steps d.-f. and the impedances Zₘₑₐₛ(ωᵢ) can be determined from the resulting current curve Iₘₑₐₛ(w) in the measuring coil.

In a further preferred embodiment, a voltage is modeled as the model output variable A_{mod}, which is induced by impressing a defined current curve, i.e. the modeled variable A_{mod} is a voltage U_{mod}. In this case, the steps of the procedure are as follows in sequence:

a. Provision of a mathematical model. The model describes the voltages U_{mod}(ω, I) in the measuring coil of a measuring circuit with a sample 4 of a sponge iron as a function of the content of metallic iron (Feₘₑₜ), iron oxides (Fe₂O₃, Fe₃O₄, or FeO), and iron carbide (Fe₃C) of the sponge iron or sample 4 thereof. For this description, the mathematical model 1 on the one hand has a determination of the impedance as a function of the permeability (μ_{eff}) and electrical conductivity (σ_{eff}) of the sponge iron or sample 4 thereof. On the other hand, the model has a determination of the permeability (μ_{eff}) and electrical conductivity (σ_{eff}) as a function of the content of metallic iron (Feₘₑₜ), iron oxides (Fe₂O₃, Fe₃O₄, or FeO), and iron carbide (Fe₃C) of the sponge iron, and as a function of the gangue fraction and the density, for which an effective medium approximation (EMA) is implemented.

b. Insertion of the effective density ρₜₒₜ of the sample 4 and the gangue fraction p_{g} of the sample 4 into the mathematical model 1.

c. Insertion of a sample 4 of the sponge iron into the magnetic circuit of the measuring apparatus.

d. Impression of a first current curve I(ω₁) into the excitation coil together with measurement/detection of a first voltage curve Uₘₑₐₛ(ω₁) as a measurement variable (Aₘₑₐₛ) in the measuring coil.

e. Impression of a second current curve I(ω₂) into the excitation coil together with measurement/detection of a second voltage curve Uₘₑₐₛ(ω₂) as a measurement variable (Aₘₑₐₛ) in the measuring coil.

f. Optional: Impression of additional current curves I(ωᵢ) into the excitation coil together with measurements/detecting of additional voltage curves Uₘₑₐₛ(ωᵢ) as measurement variables (Aₘₑₐₛ) in the measuring coil.

g. Determination of the fractions of metallic iron (Feₘₑₜ) and/or iron oxide (Fe₂O₃, Fe₃O₄, or FeO) and/or iron carbide (Fe₃C) in the sample 4 of sponge iron through implementation of a least squares estimation method using the mathematical model 1 and the measurement data for the voltage curves \vec{U}ₘₑₐₛ(ω₁), Uₘₑₐₛ(ω₂), and optionally Uₘₑₐₛ(ωᵢ).

In this case, the fractions of metallic iron (Feₘₑₜ) and/or iron oxide (Fe₂O₃, Fe₃O₄, and FeO) and/or iron carbide (Fe₃C) in the sample 4 are determined for which the sum of the deviations of the modeled voltages U_{mod} serving as the model output variable (A_{mod}) from the measured voltages Uₘₑₐₛ serving as the measurement variable (Aₘₑₐₛ) is minimal.

The mathematical model 1 that takes these into account, with approximated permeability μ_{eff} and electrical conductivity σ_{eff} for an impedance model 3 and preferably using the known parameters of effective density ρₜₒₜ and gangue fraction p_{g}, thus enables a particularly accurate determination of the content of metallic iron (Feₘₑₜ), optionally iron oxide (FeO), and optionally iron carbide (Fe₃C) of the sponge iron or sample 4 thereof.

It should be noted in general that the German expression “insbesondere” can be translated as “more particularly” in English. A feature that is preceded by “more particularly” is to be considered an optional feature, which can be omitted and does not thereby constitute a limitation, for example, of the claims. The same is true for the German expression “vorzugsweise”, which is translated as “preferably” in English.

1. A method for determining a content of at least metallic iron (Feₘₑₜ) in sponge iron or a sample thereof produced by direct reduction from iron ore, comprising the following steps:
detecting at least one measurement variable (Aₘₑₐₛ), which is dependent on at least one electromagnetic property of the sponge iron or sample thereof,
providing a mathematical model whose model output variable (A_{mod}) is described as a function of the content of at least metallic iron (Feₘₑₜ) of the sponge iron or sample thereof, wherein the mathematical model has an effective medium approximation (EMA) for the permeability (μ_{eff}) and electrical conductivity (σ_{eff}) of the sponge iron or sample thereof, and
implementing an estimation method for determining the content of at least metallic iron (Feₘₑₜ) using the detected measurement variable (Aₘₑₐₛ) and the mathematical model.

2. The method for determining content according to claim 1, wherein the mathematical model includes a gangue fraction (p_{g}) of the iron ore or sponge iron or sample thereof as a first input parameter and/or a density (ρₜₒₜ) of the sponge iron or sample thereof as a second input parameter.

3. The method for determining content according to claim 1, wherein the mathematical model is an electromagnetic model (EMM), which comprises an impedance model.

4. The method for determining content according to claim 3, wherein the impedance model comprises a modeling of at least one electrical coil with sponge iron or a sample thereof as a material of a core.

5. The method for determining content according to claim 3, wherein the model output variable (A_{mod}) is an electrical impedance (Z_{mod}(ωi)).

6. The method for determining content according to claim 3, wherein the model output variable (A_{mod}) is an electrical voltage (U_{mod}(ωᵢ), which forms taking into account a known current (I) at an electrical impedance (Z_{mod}(ωᵢ)) of the impedance model.

7. The method for determining content according to claim 3, wherein the model output variable (A_{mod}) is an electrical current (I_{mod}(ωᵢ)), which forms when a known voltage is applied at an electrical impedance (Z_{mod}(ωᵢ)) of the impedance model.

8. The method for determining content according to claim 1, comprising using the estimation method to determine the content or contents for which a deviation of the at least one detected measurement variable (Aₘₑₐₛ) from the model output variable (A_{mod}) of the mathematical model is minimal.

9. The method for determining content according to claim 1, wherein an electrical voltage (Uₘₑₐₛ) induced in a coil, which results from a magnetic field that is generated by an impressed current (I), is detected as or for the at least one measurement variable (Aₘₑₐₛ), wherein the coil has sponge iron or a sample thereof as a material of a core.

10. The method for determining content according to claim 1, wherein an electrical current (Iₘₑₐₛ) that occurs in a coil when an impressed voltage (U) is applied to the coil is detected as or for the measurement variable (Aₘₑₐₛ), wherein the coil has sponge iron or a sample thereof as a material of the core.

11. The method for determining content according to claim 1, wherein first measurement data (Aₘₑₐₛ(ω₁)) of the measurement variable (Aₘₑₐₛ) are detected by applying a first magnetic field to the sponge iron or sample thereof and second measurement data (Aₘₑₐₛ(ω₂)) of the measurement variable (Aₘₑₐₛ) are detected by applying a second magnetic field to the sponge iron or sample thereof, wherein the first and second magnetic fields change over time and have different frequencies from each other.

12. The method for determining content according to claim 1, wherein the model output variable (A_{mod}) is described as a function of a content of at least metallic iron (Feₘₑₜ) and of at least one iron oxide (Fe₂O₃, Fe₃O₄, or FeO) as well as iron carbide (Fe₃C) of the sponge iron or sample thereof, and the estimation method for determining the content of at least metallic iron (Feₘₑₜ), iron oxide (Fe₂O₃, Fe₃O₄, or FeO), and iron carbide (Fe₃C) is carried out using the detected measurement variable (Aₘₑₐₛ) and the mathematical model.

13. The method for determining content according to claim 1, wherein the at least one measurement variable (Aₘₑₐₛ) is an electrical measurement variable.

14. The method for determining content according to claim 2, wherein the density (ρₜₒₜ) is an effective density.

15. The method for determining content according to claim 6, wherein the known current (I) is an impressed current.

16. The method for determining content according to claim 7, wherein the known voltage is an impressed voltage.

17. The method for determining content according to claim 8, wherein the estimation method is a least squares estimation method.

18. The method for determining content according to claim 8, comprising using the estimation method to determine the content or contents for which the deviation of a sum of the detected measurement variables (Aₘₑₐₛ) from the model output variable (A_{mod}) of the mathematical model is minimal.
